(19) United States
(12) Patent Application Publication
(10) Pub. No.: US 2021/0296006 A1
(43) Pub. Date: Sep. 23, 2021

(54) CONTACT HISTORY CONSTRUCTION SYSTEM

(71) Applicant: THLight LOCATION SYSTEM CO, LTD., New Taipei City (TW)

(72) Inventor: Cheng-Jan CHI, NEW TAIPEI CITY (TW)

(73) Assignee: THLight LOCATION SYSTEM CO, LTD., NEW TAIPEI CITY (TW)

(21) Appl. No.: 16/896,931

(22) Filed: Jun. 9, 2020

(30) Foreign Application Priority Data
Mar. 19, 2020 (TW) 109109263

(51) Int. Cl. G16H 50/80; H04B 17/318; G16H 10/65; G06K 7/10; G06K 19/07

(52) U.S. Cl. CPC G16H 50/80; H04B 17/318; G06K 19/0723; G06K 7/10366; G16H 10/65

(57) ABSTRACT

A contact history construction system includes a plurality of detection devise and a processing device. The plurality of detection devices are used for transmitting a radio wave respectively. The radio wave includes a current time and an identity. The identity corresponds to the detection device transmitting the radio wave. The plurality of detection devices are able to receive the radio wave from each other. The detection device processes the radio wave received into a contact distance according to a signal strength of the radio wave and stores the current time, the identity and the contact distance of the radio wave received in order to construct a contact history. The processing device is coupled to any one of the detection devices and reads the contact history of the detection device coupled thereto.

### BACKGROUND OF THE INVENTION

### 1. Technical Field

The present invention relates to radio frequency (RF) detection device, and in particular, to a contact history construction system.

### 2. Description of Related Art

Spreading of infectious disease often leads to significant economic losses. For example, in case where employees are infected by a certain infectious disease in a company, to prevent the spreading of such virus, the method of plant shutdown is commonly adopted. In other words, all employees have to be suspended from works for a certain period of time. Consequently, companies can suffer significant loss due to such suspension of work of all employees.

In addition, when the health unit (such as epidemic control and prevention center) is to perform tracking on the contact history of those infected people, it often requires to spend a tremendous amount of time in the interview with all infected people, such that the tracking difficulty and work load are significantly increased.

### BRIEF SUMMARY OF THE INVENTION

In view of the aforementioned drawbacks, a contact history construction system of the present invention is able to automatically detect a contact history of a user and to record the contact history of the user. When there is a need to track the contact history of a specific user, it is able to use the contact history already recorded to find out contact personnel of high risk efficiently and accurately.

To achieve the aforementioned objective, a contact history construction system of the present invention comprises a plurality of detection devices and a processing device. The plurality of detection devices are used for transmitting a radio wave respectively. The radio wave comprises a current time and an identity. The identity corresponds to the detection device transmitting the radio wave. In addition, the plurality of detection devices are able to receive the radio wave from each other. The radio wave received from the detection device is processed into a contact distance according to a signal strength of the radio wave, and it stores the current time, the identity and the contact distance of the radio wave received in order to establish a contact history. The processing device is coupled to any one of the plurality of detection devices and reads the contact history of the detection device coupled thereto.

Accordingly, the contact history construction system of the present invention is able to use the detection device to automatically record the contact history and to read the contact history of the detection device via the processing device, thereby obtaining all contact targets within a specific period of time.

For the assembly, characteristics and method of use of the contact history construction system provided by the present invention, please refer to the subsequent embodiments described in the following. However, a person with ordinary skills in the art in the technical field of the present invention shall understand that such description and specific embodiments described in the present invention are provided to illustrate the present invention rather than to limit the scope of the claims of the present invention.

### BRIEF DESCRIPTION OF THE SEVERAL VIEWS OF THE DRAWINGS

FIG. 1 is a schematic view of the contact history construction system according to an embodiment of the present invention;

FIG. 2 is an assembly schematic view of the detection device of the present invention shown in FIG. 1; and

FIG. 3 is an assembly schematic view of the processing device of the present invention shown in FIG. 1.

### DETAILED DESCRIPTION OF THE INVENTION

The following provides a detailed description on the preferred embodiments of the present invention with respect to the assembly, application and technical effect of the contact history construction system of the present invention along with the accompanied drawings. Nevertheless, it shall be noted that the device, assembly, dimension and appearance of the contact history construction system shown in all drawings are provided to illustrate the technical features of the present invention rather than to limit the scope of the present invention.

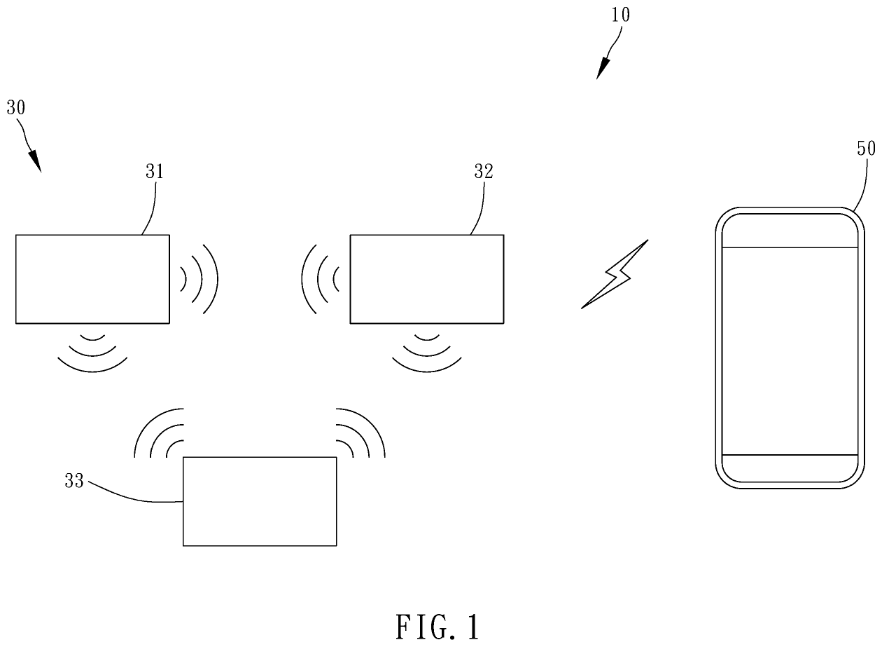

As shown in FIG. 1, the contact history construction system 10 of the present invention comprises a detection environment 30 and a processing device 50. The detection environment 30 comprises three detection devices 31, 32, 33. In an application, the detection devices 31, 32, 33 are worn on a human body or an object respectively. The detection devices 31, 32, 33 are used to generate a radio wave respectively. The radio wave comprises a current time and an identity. The detection devices 31, 32, 33 are able to receive the radio wave from each other, and construct a corresponding contact history. The processing device 50 can be coupled to any one of the detection devices 31, 32, 33 in order to read the contact history of the detection device 31, 32 or 33 coupled thereto. As shown in the drawing, the processing device 50 is coupled to the detection device 32 and receives the contact history of the detection device 32.

The following provides an example for illustration. The detection device 31 receives the radio wave transmitted by the detection devices 32, 33. Through the identity of the radio wave, the corresponding detection devices 32, 33 can be identified correctly without errors. The detection device 31 processes it into a contact distance via the signal strength of the radio wave. The signal strength comprises a signal power value. For example, when the signal strength of the radio wave transmitted by the detection device 32 is −60 dbm (decibel relative to one milliwatt), then the corresponding contact distance processed by the detection device 31 is less than 1 m; when the signal strength of the radio wave transmitted by the detection device 33 is −150 dbm, then the corresponding distance processed by the detection device 31 is greater than 2 m. Finally, the detection device 31 establishes the contact history for the detection device 31 according to the current time, the identity and the contact distance of the radio wave received.

In this embodiment, the transmission frequency of the radio wave can be ten times per second; in other embodiments, the transmission frequency can be less than ten times per second. Accordingly, under a normal condition, the radio wave transmission frequency can be extended in order to increase the time of use of the battery. However, under special condition, such as during the virus epidemic period of special virus, it can be changed to the transmission of ten times or more than ten times per second, in order to track the contact history frequently.

In another embodiment, the detection devices 31, 32, 33 are able to record the time of receiving the radio wave in the contact history.

From the above description, it can be understood that the contact distance in this embodiment is a relative distance rather than an absolute distance. Despite that the above description only explains the receiving, processing and storage operation of the detection device 31, since the receiving, process and storage of the detection devices 32 and 33 are identical to those of the detection device 31, the description for the same parts are omitted for conciseness.

In another embodiment, the detection environment 30 can have more or less quantity of devices. The quantity can be greater, such as more than three devices, or can be fewer, such as two devices. The processing device 50 can be a mobile phone, a computer, a tablet or other computing device.

As shown in FIG. 1, any one of the detection devices 31, 32, 33 of the detection environment 30 has the same assembly and functions, and they are equipped of the same communication capability. Consequently, the subsequent description provided for the detection devices 31, 32, 33 uses one of the two as an example for the illustration.

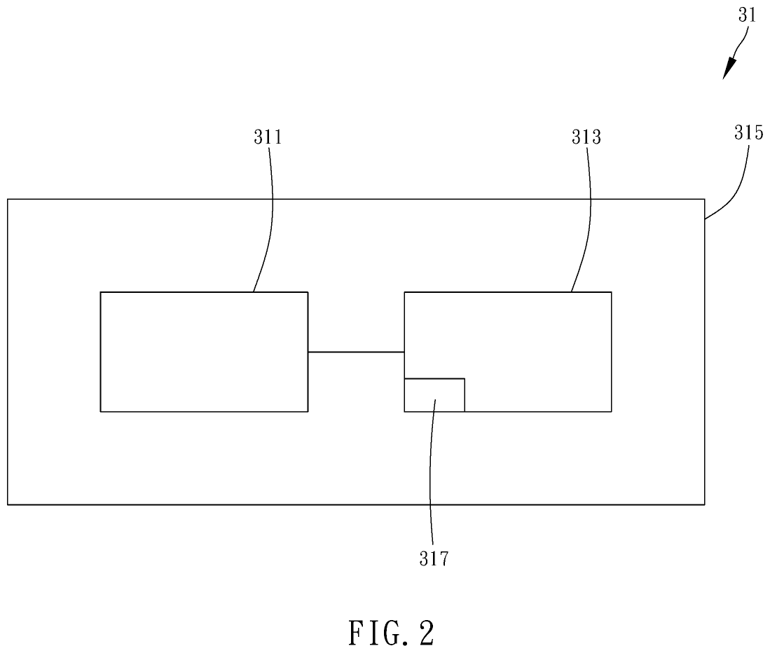

As shown in FIG. 2, the detection device 31 of the present invention comprises a battery 311, a radio frequency processing module 313 and a housing 315. The battery 311 is electrically connected to the radio frequency processing module 313 in order to supply power to the radio frequency processing module 313. The radio frequency processing module 313 can be used to transmit, receive and store radio wave. The battery 311 and the radio frequency processing module 313 are arranged inside the housing 315.

In this embodiment, the radio frequency processing module 313 uses the Bluetooth technology, to transmit and receive radio wave. The radio wave comprises a current time and an identity. The current time refers to the time when the radio frequency processing module 313 transmits the radio wave. The identity represents the detection device 31 transmitting the radio wave. In other embodiments, the radio frequency processing module 313 can also use the technologies of Radio Frequency Identification (RFID), Wi-Fi etc.

Furthermore, the radio frequency processing module 313 comprises a light emitting element 317 for generating a light according to the power level of the battery 311. When the power level of the battery 311 is low or insufficient, the light of the light emitting element 317 is weak in order to provide identification. The housing 315 comprises a light transmittance material in order to allow the light of the light emitting element 317 to penetrate through. The light transmittance material can be, such as, a transparent or semi-transparent material.

In another embodiment, the housing 315 can have different appearance of structure. Accordingly, the housing 315 is not limited to any specific appearance and structure.

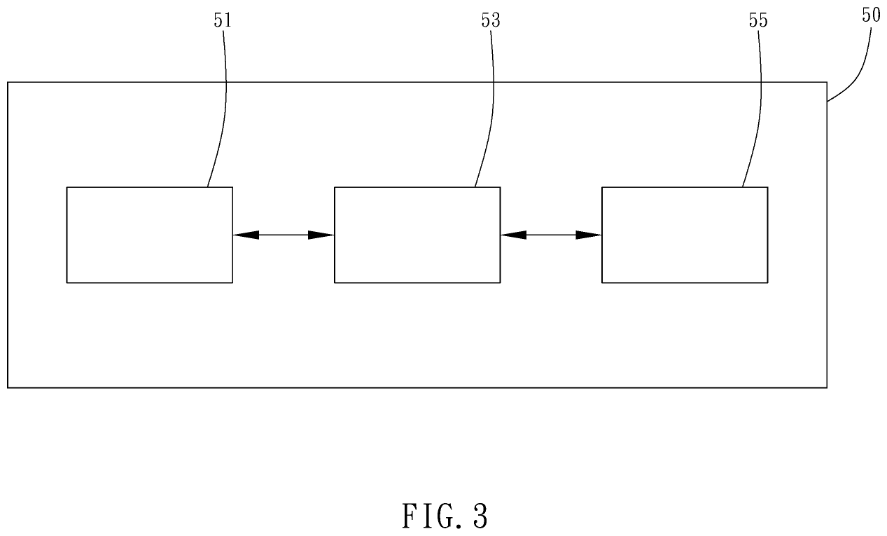

As shown in FIG. 3, the processing device 50 of the present invention comprises a receiving module 51, a processing module 53 and a display device 55. The receiving module 51 is used to receive the contact history of the detection device 31, 32, 33, and the coupling can be wired or wireless connection. The processing module 53 is electrically connected to the receiving module 51 and processes the contact history. The display device 55 is connected to the processing module 53 and is used to display the contact history. The processing module 53 is able to construct a plurality of radio wave data included in the contact history into a corresponding chart of table through the data processing method on the contact history already received, and to display it on the display device 55, in order to allow the user to observe and to track the contact history of the detection device within a specific period of time, thereby discovering the close contact, long period of time of contact or multiple times of contact of a high-risk target from the contact history.

In another embodiment, the processing device 50 uses a wireless communication technology for coupling to the detection devices 31, 32, 33; in other words, when it is coupled to a plurality of detection devices, the processing device 50 is able to synchronously receive the contact history of the detection devices 31, 32 and 33. Therefore, the coupling, receiving or reading the contact history of the processing device 50 is not limited to one detection device only. For example, when the processing device 50 detects that there are detection devices 31, 32, 33, or when the detection devices 31, 32, 33 detect that there is a processing device 50 nearby, the detection devices 31, 32, 33 are able to transmit the contact history to the processing device 50 via the encoded broadcasting technique such that the processing device 50 is able to synchronously read the contact history of the detection devices 31, 32, 33, thereby constructing a complete contact history of the detection devices 31, 32, 33, or the processing device 50 is able to transmit the contact history to other storage device, such as a Cloud or a Network Attached Storage (NAS) etc.

Accordingly, with the contact history construction system of the present invention, it is able to use the detection device to automatically detect any detection devices at the surrounding in order to construct a complete contact history of a user or an object equipped with or wearing the detection device. Furthermore, when users are confirmed to be infected by a disease, the processing device is able to read the detection devices of the corresponding users in order to obtain the contact history of such users, in order to identify the high-risk targets from the contact history, thereby further identifying targets requiring attention. As a result, in comparison to the prior arts, the present invention is able to effectively identify employees with risks in a company without the need to suspend the work of the entire company, such that the economic loss can be reduced and normal operation of the company can be maintained.

Lastly, it shall be emphasized that all devices, assemblies and applications disclosed in the aforementioned embodiments of the present invention are provided as examples for illustration only such that they shall not be treated as limitation to the scope of the present invention. Any substitution or modification with the use of other equivalent elements shall be treated to be within the scope of the claims of the present invention.

What is claimed is:

1. A contact history construction system, comprising:
a plurality of detection devices for transmitting a radio wave respectively; the radio wave comprising a current time and an identity; the identity corresponding to the detection device transmitting the radio wave; and
a processing device; wherein the plurality of detection devices receiving the radio wave transmitted from each other, processing the radio wave received by the detection devices into a contact distance according to a signal strength of the radio wave, and storing the current time, the identity and the contact distance of the radio wave received, in order to establish a contact history; the processing device coupled to any one of the plurality of detection devices and configured to read the contact history of the detection device coupled thereto.

2. The contact history construction system according to claim 1, wherein any one of the detection devices comprises a battery, a radio frequency processing module and a housing; the battery is connected to the radio frequency processing module; the battery is used for supplying power to the radio frequency processing module; the radio frequency processing module is used for transmitting, receiving and storing the radio wave; the battery and the radio frequency processing module are arranged inside the housing.

3. The contact history construction system according to claim 2, wherein the housing comprises a light transmittance material.

4. The contact history construction system according to claim 1, wherein the current time of the radio wave comprises a time when the detection device transmitting the radio wave.

5. The contact history construction system according to claim 1, wherein the signal strength comprises a signal power value.

6. The contact history construction system according to claim 1, wherein the processing device comprises a receiving module, a processing module and a display device; the receiving module is used for receiving the contact history of the detection device coupled thereto; the processing module is electrically connected to the receiving module and used for processing the contact history; the display device is connected to the processing module and used for displaying the contact history.
